(19) United States
(12) Patent Application Publication
(10) Pub. No.: US 2025/0099020 A1
(43) Pub. Date: Mar. 27, 2025

(54) COGNITIVE FUNCTION ASSESSMENT DEVICE AND COGNITIVE FUNCTION ASSESSMENT SYSTEM

(71) Applicant: Murata Manufacturing Co., Ltd., Kyoto (JP)

(72) Inventors: Hiroshi KUNIMATSU, Kyoto (JP); Yuji NIHEI, Kyoto (JP)

(21) Appl. No.: 18/969,920

(22) Filed: Dec. 5, 2024

Related U.S. Application Data
(63) Continuation of application No. PCT/JP2023/017393, filed on May 9, 2023.

(30) Foreign Application Priority Data
Jun. 6, 2022 (JP) 2022-091349

(51) Int. Cl. A61B 5/00; A61B 5/11; A61B 5/12; A61B 5/372; A61B 5/378; A61B 5/38; G16H 50/30

(52) U.S. Cl. CPC A61B 5/4088; A61B 5/11; A61B 5/121; A61B 5/372; A61B 5/378; A61B 5/38; A61B 5/7264; A61B 5/7275; A61B 5/7282; G16H 50/30; A61B 2560/0462

(57) ABSTRACT

A cognitive function assessment device includes a brain signal detection part, a response motion detection part, and a computation part. The brain signal detection part detects a brain signal of a subject occurring in response to a stimulus. The response motion detection part detects a response motion of the subject occurring in response to the stimulus and outputs a response signal. The computation part detects an event-related potential and a motor readiness potential from the brain signal, calculates a response speed from the response signal, and assesses cognitive function on the basis of the event-related potential, the motor readiness potential, and the response speed.

### CROSS REFERENCE TO RELATED APPLICATION

This is a continuation of International Application No. PCT/JP2023/017393 filed on May 9, 2023 which claims priority from Japanese Patent Application No. 2022-091349 filed on Jun. 6, 2022. The contents of these applications are incorporated herein by reference in their entireties.

### BACKGROUND ART

### Technical Field

The present disclosure relates to assessment techniques of cognitive function.

In Patent Document 1, major neurocognitive disorder is distinguished using waveforms of brain waves occurring in response to visual stimuli, auditory stimuli, and olfactory stimuli.
Patent Document 1: Japanese Unexamined Patent Application Publication No. 2019-13375

### BRIEF SUMMARY

However, in a configuration such as the one illustrated in Patent Document 1, the cognitive function is determined only using event-related potentials to stimuli. Because of this, for example, in the case of a subject having another decreased function, such as an elderly person and the like, it is difficult to make a precise assessment of cognitive function decline.

The present disclosure provides a technique to make a more precise assessment of the cognitive function of a subject.

A cognitive function assessment device of this disclosure includes a brain signal detection part, a response motion detection part, and a computation part. The brain signal detection part detects a brain signal of a subject occurring in response to a stimulus. The response motion detection part detects a response motion of the subject occurring in response to the stimulus and outputs a response signal. The computation part detects an event-related potential and a motor readiness potential from the brain signal, calculates a response speed from the response signal, and assesses cognitive function on a basis of the event-related potential, the motor readiness potential, and the response speed.

In this configuration, the cognitive function is assessed using the event-related potential to the stimulus, the motor readiness potential to the stimulus, and the response speed. Because of this, the assessment is performed using the cognitive function to the stimulus, a motor command function to the stimulus, and a motor function to the stimulus, and thus, the assessment accuracy of the cognitive function of a subject improves.

According to this disclosure, it becomes possible to make a more precise assessment of the cognitive function of a subject.

### BRIEF DESCRIPTION OF THE DRAWINGS

FIG. 1 is a functional block diagram of a cognitive function assessment system according to a first embodiment of the present disclosure.

FIG. 2 is a functional block diagram illustrating a configuration of a brain signal detection part.

FIG. 3 is a diagram schematically illustrating a wearing state of a detection gear for a cognitive function subject.

FIG. 4 is a functional block diagram illustrating a configuration of a computation part.

FIG. 5A and FIG. 5B are diagrams illustrating assessment concepts of cognitive function.

FIG. 6 is a flowchart illustrating one example of a cognitive function assessment method according to the first embodiment.

FIG. 7 is a flowchart illustrating one example of a cognitive function assessment.

FIG. 8 is a flowchart illustrating one example of a classification method and an assessment method of cognitive function decline.

FIG. 9 is a functional block diagram of a cognitive function assessment system according to a second embodiment of the present disclosure.

FIG. 10 is a flowchart illustrating one example of a cognitive function assessment method according to the second embodiment.

FIG. 11 is a functional block diagram of a cognitive function assessment system according to a third embodiment of the present disclosure.

FIG. 12 is a flowchart illustrating one example of a cognitive function assessment method according to the third embodiment.

FIG. 13 is a flowchart illustrating one example of a cognitive function assessment method according to a fourth embodiment.

FIG. 14 is a flowchart illustrating one example of a cognitive function assessment method according to the fourth embodiment.

FIG. 15 is a flowchart illustrating one example of a cognitive function assessment method according to a fifth embodiment.

### DETAILED DESCRIPTION

### First Embodiment

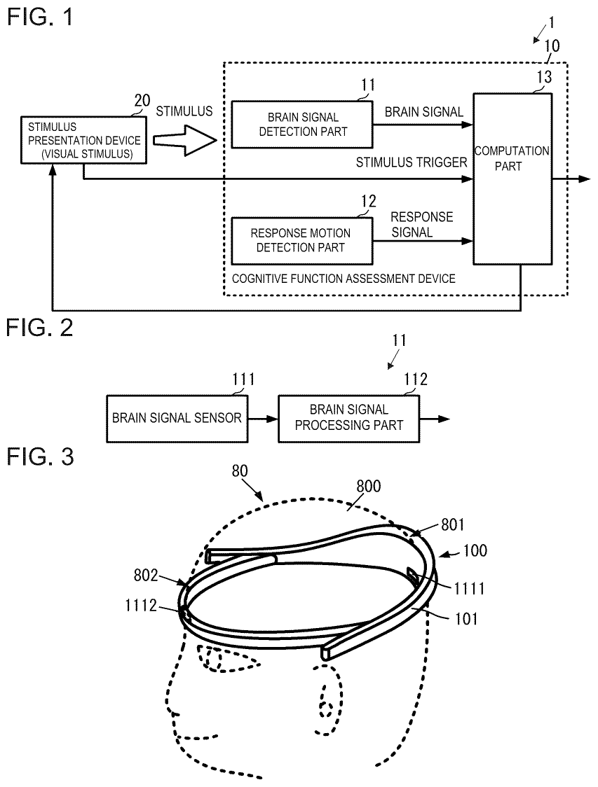

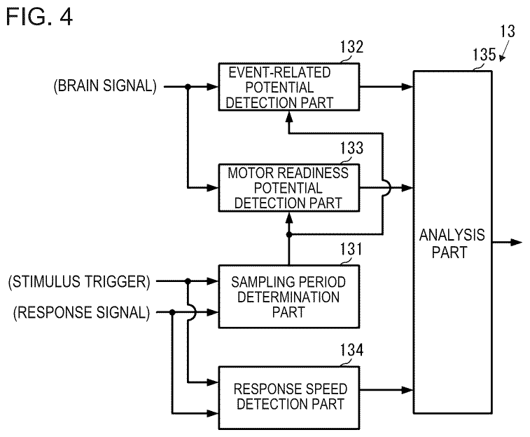

A cognitive function assessment system according to the first embodiment of the present disclosure is now described with reference to the drawings. FIG. 1 is a functional block diagram of the cognitive function assessment system according to the first embodiment of the present disclosure. FIG. 2 is a functional block diagram illustrating a configuration of a brain signal detection part. FIG. 3 is a diagram schematically illustrating a wearing state of a detection gear for a cognitive function subject. FIG. 4 is a functional block diagram illustrating a configuration of a computation part.

### (Configuration of Cognitive Function Assessment System 1)

As illustrated in FIG. 1, the cognitive function assessment system 1 includes a cognitive function assessment device 10 and a stimulus presentation device 20. The cognitive function assessment device 10 includes a brain signal detection part 11, a response motion detection part 12, and a computation part 13.

### (Stimulus Presentation Device 20)

The stimulus presentation device 20 presents visual stimuli to a subject. For example, the stimulus presentation device 20 is a display device capable of displaying predetermined images.

The stimulus presentation device 20 displays, as visual stimuli, a first image and a second image that have different shapes (for example, two types of circles having different sizes) at predetermined cycles. In this case, the displaying frequency of the first image is different from the displaying frequency of the second image.

For example, the first image is displayed more frequently while the second image is displayed less frequently. More specifically, the first image is routinely displayed every second, but at a specific timing, the second image is displayed in place of the first image.

For example, upon receipt of a command to start the presentation of visual stimuli from the computation part 13 of the cognitive function assessment device 10, the stimulus presentation device 20 presents visual stimuli. Note that the stimulus presentation device 20 does not necessarily receive the command to start the presentation of visual stimuli from the computation part 13. For example, another functional part or the like, which is not illustrated, may cause visual stimuli to be presented so as to synchronize with each process of the computation part 13 or each sampling period.

### (Brain Signal Detection Part 11)

As illustrated in FIG. 2, the brain signal detection part 11 includes a brain signal sensor 111 and a brain signal processing part 112. The brain signal sensor 111 is realized, for example, using a known sensor that can acquire a brain signal.

The brain signal sensor 111 is worn by a subject, for example, using a detection gear 100 such as the one illustrated in FIG. 3. The detection gear 100 includes a head band 101, a brain signal sensor 1111, and a brain signal sensor 1112. The brain signal sensor 1111 and the brain signal sensor 1112 correspond to the brain signal sensor 111 of FIG. 2. Note that any number of brain signal sensors may be included in the detection gear 100.

The head band 101 is made of a belt-like base material. For example, the head band 101 is stretchable. The head band 101 is put on a head 800 of a subject 80. The brain signal sensor 1111 is installed on the inner side of the head band 101 on the back-of-head 801 side. The brain signal sensor 1112 is installed on the inner side of the head band 101 on the front-of-head 802 side. The brain signal sensor 1111 and the brain signal sensor 1112 acquire brain signals of the subject 80 and output the acquired brain signals to the brain signal processing part 112.

The brain signal processing part 112 is realized, for example, using an electronic circuit, an IC, or the like. The brain signal processing part 112 performs a filtering process, an amplifying process, and the like on the brain signals acquired by the brain signal sensor 111 (the brain signal sensor 1111 and the brain signal sensor 1112). The brain signal processing part 112 outputs, to the computation part 13, a brain signal subjected to a predetermined process. Note that the detection gear 100 is not limited to the one disclosed in FIG. 3, and for example, may alternatively be one built into a head mount display (HMD), a VR (Virtual Realty) device, or the like.

### (Response Motion Detection Part 12)

For example, the response motion detection part 12 is realized using physical buttons, a touch panel, an operation input device (for example, a mouse or the like) installed in the cognitive function assessment device 10, or the like. That is to say, the response motion detection part 12 is realized using a member that can detect a motion caused by the subject 80 when a cognition target event occurs.

The response motion detection part 12 detects an input timing (operation timing) of a response motion by the subject 80 and calculates a response signal. The response motion detection part 12 outputs the response signal to the computation part 13.

### (Computation Part 13)

As illustrated in FIG. 4, the computation part 13 includes a sampling period determination part 131, an event-related potential detection part 132, a motor readiness potential detection part 133, a response speed detection part 134, and an analysis part 135. The computation part 13 is realized using an electronic circuit, an IC, an MPU, or the like.

A stimulus trigger from the stimulus presentation device 20 or a response signal from the response motion detection part 12 is input to the sampling period determination part 131. The stimulus trigger is a trigger that indicates the timing at which a response target image (vision) is presented. The sampling period determination part 131 determines a sampling period for detecting event-related potentials and a sampling period for detecting motor readiness potentials.

The sampling period determination part 131 sets the sampling period for detecting event-related potentials in the event-related potential detection part 132. The sampling period determination part 131 sets the sampling period for detecting motor readiness potentials in the motor readiness potential detection part 133.

The brain signal is input to the event-related potential detection part 132. The event-related potential detection part 132 detects event-related potentials related to visual stimuli (for example, P300) from the waveform of the brain signal or the like in the sampling period for detecting the event-related potentials of visual stimuli. The event-related potential detection part 132 outputs the detected event-related potentials to the analysis part 135.

The brain signal is input to the motor readiness potential detection part 133. The motor readiness potential detection part 133 detects the motor readiness potential from a waveform of the brain signal or the like in the sampling period for detecting the motor readiness potentials. The motor readiness potential detection part 133 outputs the motor readiness potentials to the analysis part 135.

The stimulus trigger and the response signal are input to the response speed detection part 134. The response speed detection part 134 detects a response speed using the time difference between the timing of the stimulus trigger and the timing of the response signal. The response speed detection part 134 outputs the response speed to the analysis part 135. Note that instead of using the stimulus trigger, the response speed detection part 134 may detect the response speed using the time difference between the timing of the motor readiness potential and the timing of the response signal.

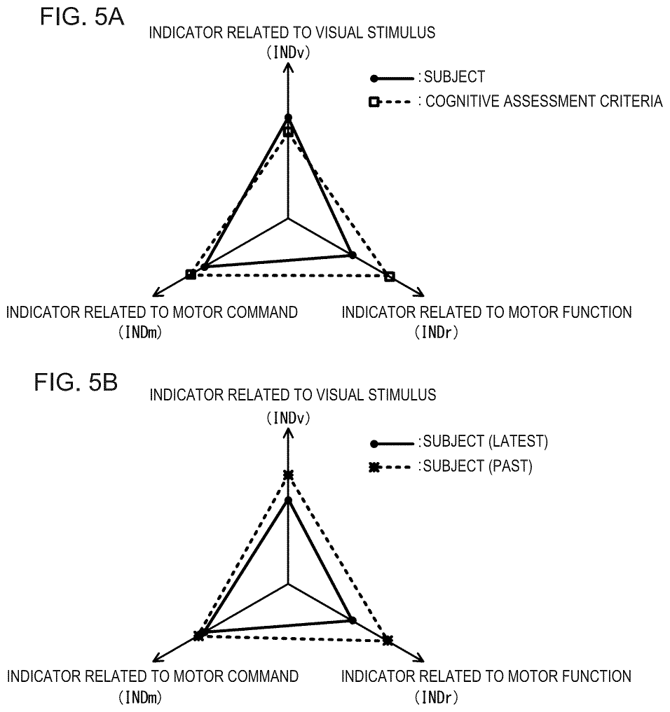

The analysis part 135 assesses cognitive function using the event-related potential, the motor readiness potential, and the response speed. More specifically, for example, the analysis part 135 assesses the cognitive function using the techniques which will be described below. FIG. 5A and FIG. 5B are diagrams illustrating assessment concepts of cognitive function.

The analysis part 135 calculates an indicator INDv related to cognition of the visual stimulus using the event-related potential. The indicator INDv is calculated using a waveform, an amplitude, or the like of the event-related potential. For example, the indicator INDv is an indicator whose lower value means a lower cognitive function to the visual stimulus.

The analysis part 135 calculates an indicator INDm related to the motor readiness to the stimulus using the motor readiness potential. The indicator INDm is calculated using a waveform, an amplitude, a timing of occurrence, or the like of the motor readiness potential. For example, the indicator INDm is an indicator whose lower value means a lower motor readiness ability to the stimulus.

The analysis part 135 calculates an indicator INDr related to the motor function to the stimulus using the response speed. The indicator INDr is calculated using the speed of the response speed. For example, the indicator INDr is an indicator whose lower value means a lower motor ability to the stimulus.

The analysis part 135 assesses the cognitive function using the indicator INDv, the indicator INDm, and the indicator INDr.

For example, with regard to the indicator INDv, the indicator INDm, and the indicator INDr, the analysis part 135 compares each indicator of the subject 80 with each indicator of cognitive assessment criteria. The analysis part 135 assesses the cognitive function using this comparison result.

For example, in the case of FIG. 5A, the indicator INDv of the subject 80 is higher than the indicator INDv of the cognitive assessment criteria. The indicator INDm of the subject 80 is nearly equal to the indicator INDm of the cognitive assessment criteria. The indicator INDr of the subject 80 is lower than the indicator INDr of the cognitive assessment criteria. In such a case, the analysis part 135 assesses that the cognitive function to stimuli is normal but the motor system ability to stimuli is low. Because of this, for example, the analysis part 135 assesses that the cognitive function is unproblematic but the motor system ability is lower than a general level.

On the other hand, when the analysis part 135 detects that the indicator INDr is lower than the cognitive assessment criterion, the analysis part 135 assesses that the cognitive function is low. Because of this, for example, the analysis part 135 assesses that there is a possibility of major neurocognitive disorder.

Further, for example, with regard to the indicator INDv, the indicator INDm, and the indicator INDr, the analysis part 135 compares each of latest indicators of the subject 80 with each of past indicators of the subject 80. The analysis part 135 assesses the cognitive function decline using this comparison result. That is to say, the analysis part 135 assesses the cognitive function decline using changes of the indicators over a plurality of different time points (in other words, the change in the cognitive function).

For example, in the case of FIG. 5B, the latest indicator INDv of the subject 80 is lower than the past indicator INDv of the subject 80. The latest indicator INDm of the subject 80 is nearly equal to the past indicator INDm of the subject 80. The latest indicator INDr of the subject 80 is lower than the past indicator INDr. In such a case, the analysis part 135 assesses that the cognitive function and the motor ability are decreased.

On the other hand, when the analysis part 135 detects that the latest indicator INDv is nearly equal to or higher than the past indicator INDv, the analysis part 135 assesses that the cognitive function is not decreased. Further, in this case, if the latest indicator INDm and the latest indicator INDr are lower than the corresponding past indicators, the analysis part 135 assesses that the cognitive function is not decreased but the motor system ability is decreased.

As described above, the cognitive function assessment system 1 and the cognitive function assessment device 10 can assess the cognitive function using a plurality of indicators including one relating to the motor system for the visual stimulus. Because of this, the cognitive function assessment system 1 and the cognitive function assessment device 10 can make a more precise assessment of the cognitive function of a subject by further considering the motor system ability of the subject.

Note that the cognitive function assessment device 10 may estimate the Mini-Mental State Examination (MMSE) score using a combination of the event-related potential, the motor readiness potential, and the response speed or a combination of the indicator INDv, the indicator INDm, and the indicator INDr, which are described above. For example, the cognitive function assessment device 10 learns in advance, as the feature value, the combination of the event-related potential, the motor readiness potential, and the response speed or the combination of the indicator INDv, the indicator INDm, and the indicator INDr in connection with the point of the MMSE score. The cognitive function assessment device 10 estimates the point of the MMSE score by entering the combination of the latest event-related potential, the latest motor readiness potential, and the latest response speed or the combination of the latest indicator INDv, the latest indicator INDm, and the latest indicator INDr into an estimation equation derived from this learning.

Further, although it is not illustrated, the cognitive function assessment device 10 may include a display device and display an assessment result.

### (Cognitive Function Assessment Method)

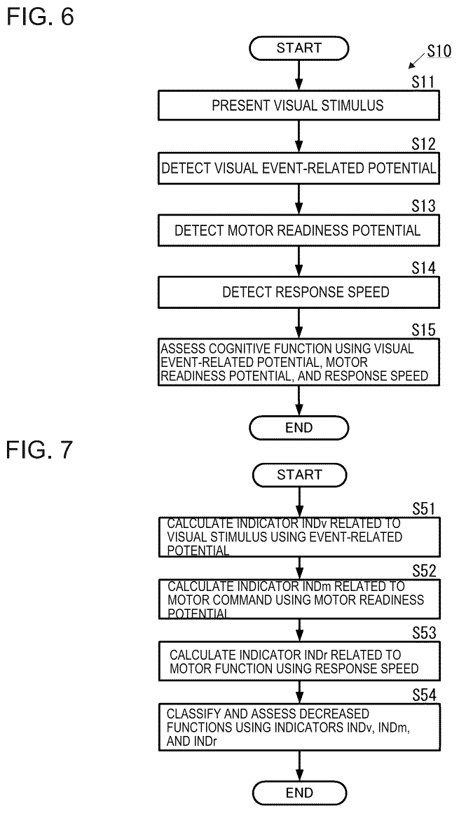

FIG. 6 is a flowchart illustrating one example of a cognitive function assessment method according to the first embodiment. Note that with regard to the processes of FIG. 6, for the parts similar to the description of the configuration described above, the descriptions are omitted.

The stimulus presentation device 20 presents visual stimuli (S11). The computation part 13 of the cognitive function assessment device 10 detects the visual event-related potential (S12). The computation part 13 of the cognitive function assessment device 10 detects the motor readiness potential (S13). The computation part 13 of the cognitive function assessment device 10 detects the response speed (S14).

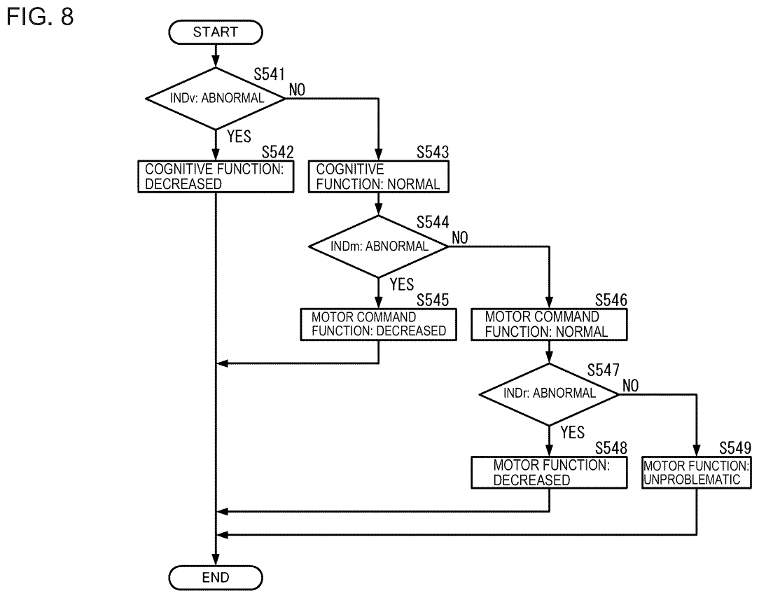

The computation part 13 of the cognitive function assessment device 10 assesses the cognitive function using the visual event-related potential, the motor readiness potential, and the response speed (S15). Specifically, the computation part 13 performs, for example, processes illustrated in FIG. 7 and FIG. 8. FIG. 7 is a flowchart illustrating one example of the cognitive function assessment. FIG. 8 is a flowchart illustrating one example of a classification method and an assessment method of cognitive function decline.

The computation part 13 calculates the indicator INDv related to the visual stimulus using the event-related potential (S51). The computation part 13 calculates the indicator INDm related to the motor command using the motor readiness potential (S52). The computation part 13 calculates the indicator INDr related to the motor function using the response speed (S53). Note that the order of calculation of the indicator INDv, the indicator INDm, and the indicator INDr is not limited to this, and the indicators may be calculated in parallel to each other.

The computation part 13 classifies and assesses decreased functions using the indicator INDv, the indicator INDm, and the indicator INDr (S54). More specifically, the computation part 13 performs, for example, processes illustrated in FIG. 8.

When the indicator INDv related to the visual stimulus is abnormal (S541: YES), the computation part 13 classifies the cognitive function as being in a decreased state or a low state (S542). The situation where the indicator INDv is abnormal means, for example, a case where the indicator INDv is lower than a criterion value indicating a low cognitive function.

When the indicator INDv is normal (S541: NO), the computation part 13 classifies the cognitive function as being in a normal state (S543). The situation where the indicator INDv is normal means, for example, a case where the indicator INDv is higher than the criterion value indicating the low cognitive function. Note that as is the case with the indicator INDv, in a specific classification, a preset criterion value is used for each of the following indicators INDm and INDr. Therefore, for the following indicators INDm and INDr, the description regarding the classification method using the criterion value is omitted.

When the indicator INDm related to the motor readiness is abnormal (S544: YES), the computation part 13 classifies the motor command function as being in a decreased state or a low state (S545). That is to say, the computation part 13 classifies the cognitive function as normal but classifies the motor command function as being in the low state.

When the indicator INDm is normal (S544: NO), the computation part 13 classifies the motor command function as being in a normal state (S546).

When the indicator INDr related to the motor function is abnormal (S547: YES), the computation part 13 classifies the motor function as being in a decreased state or a low state (S548). That is to say, the computation part 13 classifies the cognitive function and the motor command function (that is, brain functions) as normal but classifies the motor function as being in the low state.

When the indicator INDr related to the motor function is normal (S547: NO), the computation part 13 classifies the motor function as being in a normal state (S549). That is to say, the computation part 13 classifies the cognitive function, the motor command function, and the motor function as unproblematic.

As described above, the cognitive function assessment device 10 can classify and assess the cognitive function by combining the indicators related to the motion with the indicator using the event-related potential. According to this, the cognitive function assessment device 10 can make a more precise assessment of the cognitive function.

### Second Embodiment

A cognitive function assessment system according to the second embodiment of the present disclosure is now described with reference to the drawings. A cognitive function assessment system 1A according to the second embodiment is different from the cognitive function assessment system 1 according to the first embodiment in that the cognitive function assessment system 1A uses the auditory stimulus while the cognitive function assessment system 1 uses the visual stimulus. The remaining basic configuration and process of the cognitive function assessment system 1A are similar to those of the cognitive function assessment system 1, and the descriptions regarding the similar parts are omitted.

### (Configuration and Process of Cognitive Function Assessment System 1A)

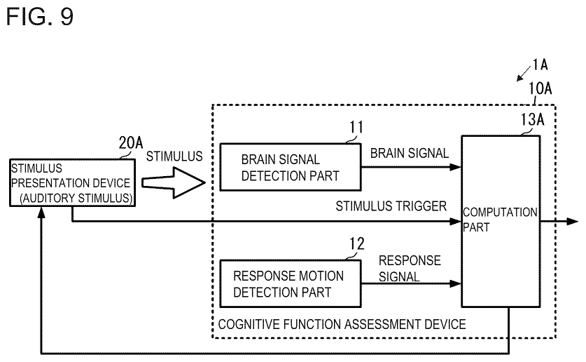

FIG. 9 is a functional block diagram of the cognitive function assessment system according to the second embodiment of the present disclosure. The cognitive function assessment system 1A according to the second embodiment includes a cognitive function assessment device 10A and a stimulus presentation device 20A.

The stimulus presentation device 20A presents auditory stimuli. For example, the stimulus presentation device 20A is a loudspeaker capable of emitting predetermined sounds.

The stimulus presentation device 20A emits, as auditory stimuli, a first sound and a second sound that have different frequencies at predetermined cycles. In this case, the sound emitting frequency of the first sound is different from the sound emitting frequency of the second sound.

For example, the first sound is emitted more frequently while the second sound is emitted less frequently. More specifically, the first sound is routinely emitted every second, but at a specific timing, the second sound is emitted in place of the first sound.

For example, upon receipt of a command to start the presentation of auditory stimuli from a computation part 13A of the cognitive function assessment device 10A, the stimulus presentation device 20A presents auditory stimuli.

The computation part 13A of the cognitive function assessment device 10A is different from the computation part 13 according to the first embodiment in that event-related potentials to auditory stimuli are used as the event-related potentials. The remaining basic configuration and process of the computation part 13A are similar to those of the computation part 13, and the descriptions thereof are omitted except the part where a new description is needed.

The event-related potential detection part 132 of the computation part 13A (see FIG. 8 and FIG. 4) detects event-related potentials related to auditory stimuli (for example, MMN) from the waveform of the brain signal or the like in the sampling period for detecting the event-related potentials of auditory stimuli. The event-related potential detection part 132 outputs detected event-related potentials to the analysis part 135.

The analysis part 135 calculates an indicator INDh related to the auditory stimulus using the event-related potentials of auditory stimuli. The analysis part 135 assesses the cognitive function using the indicator INDh related to the auditory stimulus, the indicator INDm related to the motor command, and the indicator INDr related to the motor function.

### (Cognitive Function Assessment Method)

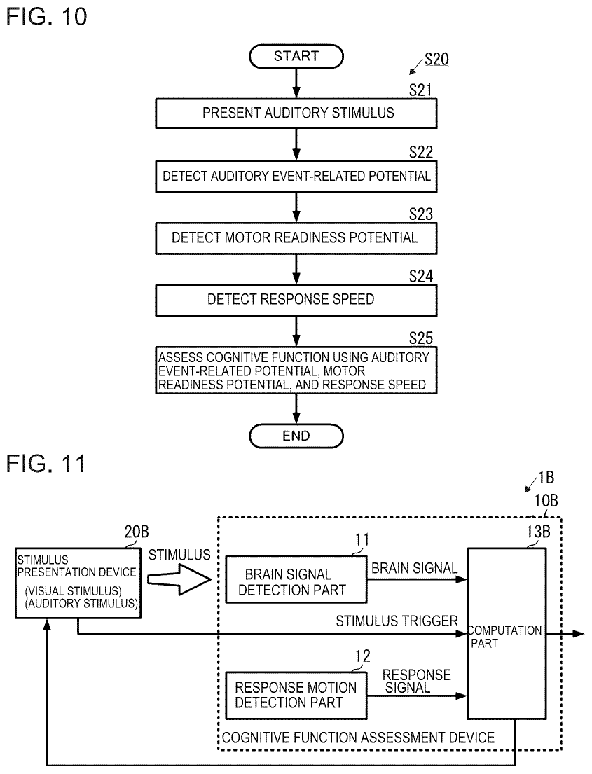

FIG. 10 is a flowchart illustrating one example of a cognitive function assessment method according to the second embodiment.

The stimulus presentation device 20A presents auditory stimuli (S21). The computation part 13A of the cognitive function assessment device 10A detects the auditory event-related potential (S22). The computation part 13A detects the motor readiness potential (S23). The computation part 13A detects the response speed (S24). The computation part 13A assesses the cognitive function using the auditory event-related potential (indicator INDh), the motor readiness potential (indicator INDm), and the response speed (indicator INDr) (S25).

As described above, the cognitive function assessment system 1A according to the second embodiment assesses the cognitive function using the auditory stimulus. As is the case with the cognitive function assessment system 1, also in this case, the cognitive function assessment system 1A can make a more precise assessment of the cognitive function.

### Third Embodiment

A cognitive function assessment system according to the third embodiment of the present disclosure is now described with reference to the drawings. The cognitive function assessment system 1 according to the first embodiment uses the visual stimulus, and the cognitive function assessment system 1A according to the second embodiment uses the auditory stimulus. On the other hand, a cognitive function assessment system 1B according to the third embodiment is different from the cognitive function assessment systems 1 and 1A in selectively using the visual stimulus and the auditory stimulus. The remaining basic configuration and process of the cognitive function assessment system 1B are similar to those of the cognitive function assessment systems 1 and 1A, and the descriptions regarding the similar parts are omitted.

### (Configuration and Process of Cognitive Function Assessment System 1B)

FIG. 11 is a functional block diagram of the cognitive function assessment system according to the third embodiment of the present disclosure. The cognitive function assessment system 1B according to the third embodiment includes a cognitive function assessment device 10B and a stimulus presentation device 20B.

The stimulus presentation device 20B selectively presents visual stimuli and auditory stimuli.

A computation part 13B of the cognitive function assessment device 10B makes a comprehensive assessment using an assessment result (assessment result of auditory cognitive function) including the event-related potentials to auditory stimuli (auditory event-related potentials) and an assessment result (assessment result of visual cognitive function) including the event-related potentials to visual stimuli (visual event-related potentials).

For the comprehensive assessment, for example, the computation part 13B compares the assessment result of auditory cognitive function with the assessment result of visual cognitive function and takes one of the assessment results that indicates the lower cognitive function as a comprehensive assessment. Alternatively, the computation part 13B translates the assessment result of auditory cognitive function and the assessment result of visual cognitive function into numerical points and takes an average value of these numerical points as a comprehensive assessment. Still alternatively, the computation part 13B may assign a weight to one of the assessment result of auditory cognitive function and the assessment result of visual cognitive function, make an assessment with one of the assessment results having a higher weight, and take a result of this assessment as a comprehensive assessment.

As described above, the cognitive function assessment system 1B makes a comprehensive assessment using a plurality of assessment results using different event-related potentials. Because of this, the cognitive function assessment device 10B can make a more precise assessment of the cognitive function.

### (Cognitive Function Assessment Method)

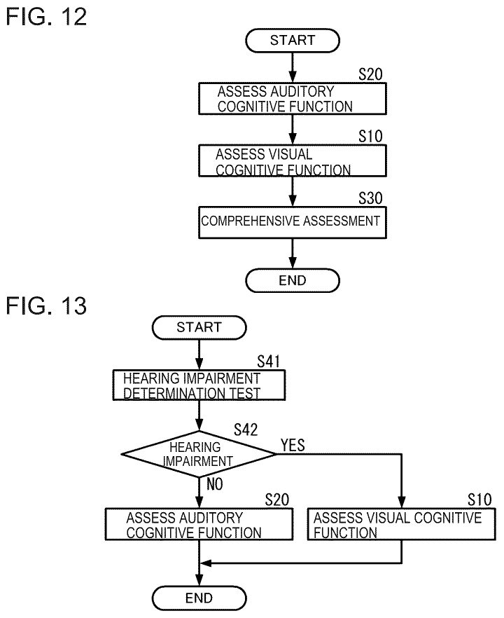

FIG. 12 is a flowchart illustrating one example of a cognitive function assessment method according to the third embodiment.

The cognitive function assessment device 10B assesses the auditory cognitive function (S20: see FIG. 10). The cognitive function assessment device 10B assesses the visual cognitive function (S10: see FIG. 6). The cognitive function assessment device 10B makes a comprehensive assessment using the assessment result of the auditory cognitive function and the assessment result of the visual cognitive function (S30).

### Fourth Embodiment

A cognitive function assessment system according to the fourth embodiment of the present disclosure is now described with reference to the drawings. The cognitive function assessment system according to the fourth embodiment has a configuration similar to that of the cognitive function assessment system 1B according to the third embodiment but uses a different assessment method. Accordingly, in the following section, the cognitive function assessment system according to the fourth embodiment is described only on points different from the cognitive function assessment system according to the third embodiment, and descriptions regarding the remaining points are omitted.

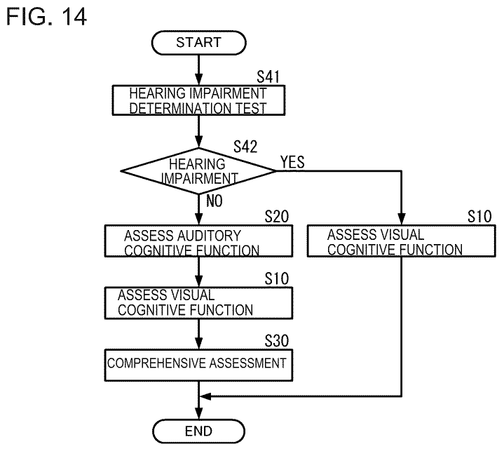

FIG. 13 and FIG. 14 are flowcharts each illustrating one example of a cognitive function assessment method according to the fourth embodiment.

In the case of FIG. 13, the cognitive function assessment system performs a hearing impairment determination test (S41). Specifically, the stimulus presentation device emits a sound for detecting hearing impairment. The sound for detecting hearing impairment may be the same as or different from the sound for presenting the auditory stimulus. The cognitive function assessment device detects a response of the subject 80 to the sound for detecting hearing impairment. The detection of this response can be achieved by using a method similar to the one used for detecting a response to the auditory stimulus.

When the cognitive function assessment system determines that the subject 80 does not have hearing impairment (S42: NO), the cognitive function assessment system assesses the auditory cognitive function (S20). Specifically, the cognitive function assessment device sends, to the stimulus presentation device, a command to present the auditory stimulus, and the stimulus presentation device presents the auditory stimulus. The cognitive function assessment device assesses the cognitive function using the auditory stimulus.

When the cognitive function assessment system determines that the subject 80 has hearing impairment (S42: YES), the cognitive function assessment system assesses the visual cognitive function (S10). Specifically, the cognitive function assessment device sends, to the stimulus presentation device, a command to switch the auditory stimulus to the visual stimulus and present the visual stimulus, and the stimulus presentation device presents the visual stimulus. The cognitive function assessment device assesses the cognitive function using the visual stimulus.

Note that the determination of whether or not the subject 80 has hearing impairment can be achieved, for example, by repeating the emission of a sound for detecting hearing impairment and the detection of sound recognition using the brain signal or the detection of response input predetermined times and further detecting a predetermined number of failures to detect the sound recognition or a predetermined number of failures to detect the response input.

In the case of FIG. 14, the cognitive function assessment system performs the same processes up to the determination of hearing impairment and performs the same process when hearing impairment is detected as in the case of FIG. 13. In the case of FIG. 14, the cognitive function assessment system performs the following processes when no hearing impairment is detected.

The cognitive function assessment system assesses the auditory cognitive function and the visual cognitive function (S20 and S10). The cognitive function assessment system makes a comprehensive assessment using the assessment of the auditory cognitive function and the assessment of the visual cognitive function (S30).

As described above, the cognitive function assessment system can make a more precise assessment of the cognitive function by considering the physical state of a subject.

### Fifth Embodiment

A cognitive function assessment system according to the fifth embodiment of the present disclosure is now described with reference to the drawings. The cognitive function assessment system according to the fifth embodiment is different from the cognitive function assessment system according to the fourth embodiment, which performs the processes illustrated in FIG. 14, in that an initial test is added and a result of the initial test is reflected in the comprehensive assessment. The processes of the cognitive function assessment system according to the fifth embodiment are similar to those of the cognitive function assessment system according to the fourth embodiment, and the descriptions regarding the similar parts are omitted.

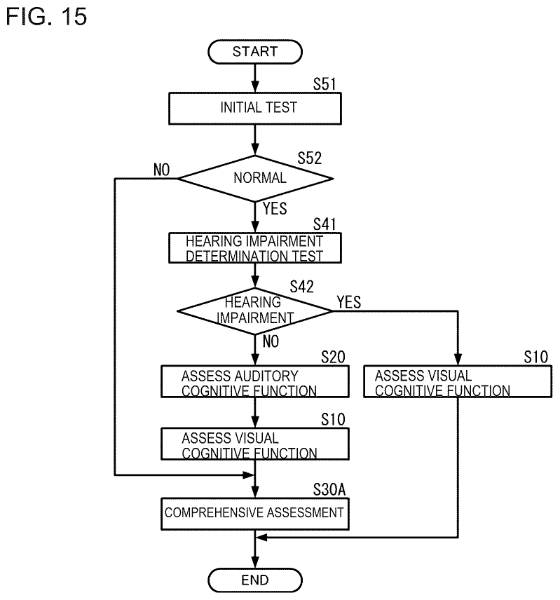

FIG. 15 is a flowchart illustrating one example of a cognitive function assessment method according to the fifth embodiment.

The cognitive function assessment system performs the initial test (S51). The initial test includes, for example, at least one of a test to detect the wearing state and a test to measure the degree of concentration of the subject 80.

In the test to detect the wearing state of the detection gear 100, for example, the cognitive function assessment system measures the amplitude of the brain signal to detect the wearing state. For example, when the amplitude is less than a predetermined threshold value, the cognitive function assessment system determines that the wearing state is imperfect, and when the amplitude is greater than or equal to the predetermined threshold value, the cognitive function assessment system determines that the wearing state is normal. Note that in the case where the wearing state is imperfect, the cognitive function assessment system may notify the subject 80, an evaluator, or the like of the imperfect wearing state. Because of this, for example, it becomes possible to provide support for correcting the wearing state.

In the test to measure the degree of concentration, for example, the cognitive function assessment system analyzes the brain signal using frequency analysis and determines the state of alpha wave. The cognitive function assessment system determines the state of the subject 80 as not being able to concentrate or the like on the basis of the state of alpha wave (brain signal having a specific frequency or specific frequencies). For example, the cognitive function assessment system determines that the subject 80 is normal when the alpha wave is within a normal value range and estimates that the subject 80 is abnormal when the alpha wave is out of the normal value range. Here, the term “abnormal” means, for example, that there is a possibility of cognitive function decline.

When the state of the subject 80 or the wearing state is normal (S52: YES), the cognitive function assessment system proceeds to the hearing impairment determination test (S41).

When the state of the subject 80 is abnormal (S52: NO), the cognitive function assessment system reflects this determination result in the comprehensive assessment (S30A). Note that when the state of the subject 80 is abnormal, the cognitive function assessment system may stop assessing the cognitive function (impossible to assess).

As described above, the cognitive function assessment system can make a more precise assessment of the cognitive function by considering the wearing state of the detection gear 100 or the physical state and the psychological state of a subject.

<1> A cognitive function assessment device comprising:
a brain signal detection part that detects a brain signal of a subject occurring in response to a stimulus;
a response motion detection part that detects a response motion of the subject occurring in response to the stimulus and outputs a response signal; and
a computation part that detects an event-related potential and a motor readiness potential from the brain signal, calculates a response speed from the response signal, and assesses cognitive function on a basis of the event-related potential, the motor readiness potential, and the response speed.

<2> The cognitive function assessment device of <1>, wherein
the computation part
   calculates a first indicator related to stimulus cognition using the event-related potential,
   calculates a second indicator related to a motor command using the motor readiness potential,
   calculates a third indicator related to a motor function using the response speed, and
   assesses the cognitive function using the first indicator, the second indicator, and the third indicator.

<3> The cognitive function assessment device of <1> or <2>, wherein
the computation part
   assesses the cognitive function at a plurality of time points that are different from one another, and
   assesses a decline in the cognitive function using a change in the cognitive function over the plurality of time points.

<4> The cognitive function assessment device of any one of <1> to <3>, wherein
the computation part estimates a MMSE score using an assessment result of the cognitive function.

<5> A cognitive function assessment system comprising:
the cognitive function assessment device of any one of <1> to <4>; and
a stimulus presentation device that presents the stimulus to the subject.

<6> The cognitive function assessment system of <5>, wherein
the stimulus presentation device presents a visual stimulus, and
the computation part detects, as the event-related potential, an event-related potential caused by the visual stimulus.

<7> The cognitive function assessment system of <5>, wherein
the stimulus presentation device presents an auditory stimulus, and
the computation part detects, as the event-related potential, an event-related potential caused by the auditory stimulus.

<8> The cognitive function assessment system of <5>, wherein
the stimulus presentation device selectively presents a visual stimulus and an auditory stimulus, and
the computation part
   detects, as the event-related potential, at least one of an event-related potential caused by the visual stimulus and an event-related potential caused by the auditory stimulus, and
   assesses the cognitive function using the event-related potential caused by the visual stimulus or the event-related potential caused by the auditory stimulus.

<9> The cognitive function assessment system of <8>, wherein
the stimulus presentation device presents a sound for detecting hearing impairment before presenting the auditory stimulus,
the computation part
   detects hearing impairment at least using the brain signal, and
   when the hearing impairment is detected, sends a command to present the visual stimulus to the stimulus presentation device,
the stimulus presentation device presents the visual stimulus, and
the computation part assesses the cognitive function using the event-related potential caused by the visual stimulus.

<10> The cognitive function assessment system of any one of <5> to <9>, wherein
the computation part
   estimates, before a stimulus is presented, a possibility of cognitive function decline using, in the brain signal, a brain signal having a specific frequency, and
   assesses the cognitive function further using a result of estimation.

### REFERENCE SIGNS LIST

1, 1A, 1B Cognitive function assessment system
10, 10A, 10B Cognitive function assessment device
11 Brain signal detection part
12 Response motion detection part
13, 13A, 13B Computation part
20, 20A, 20B Stimulus presentation device
80 Subject
100 Detection gear
101 Head band
111, 1111, 1112 Brain signal sensor
112 Brain signal processing part
131 Sampling period determination part
132 Event-related potential detection part
133 Motor readiness potential detection part
134 Response speed detection part
135 Analysis part
800 Head
801 Back-of-head
802 Front-of-head

1. A cognitive function assessment device comprising:
a brain signal detection sensor configured to detect a brain signal of a subject occurring in response to a stimulus;
a response motion detection input device configured to detect a response motion of the subject occurring in response to the stimulus and to output a response signal; and
processing circuitry configured to:
detect an event-related potential from the brain signal,
determine a response speed from a time period from the stimulus to the response signal,
determine a first indicator related to stimulus cognition using the event-related potential,
determine a third indicator related to a motor function using the response speed,
assess the cognitive function as being decreased when the first indicator is abnormal, and
assess the cognitive function as being normal but the motor function as being in a low state when the first indicator is normal and the third indicator is abnormal.

2. The cognitive function assessment device according to claim 1, wherein the processing circuitry is further configured to:
detect a motor readiness potential from the brain signal,
determine a second indicator related to a motor command using the motor readiness potential, and
assess the cognitive function as being normal but a motor command function as being in a low state when the first indicator is normal and the second indicator is abnormal.

3. The cognitive function assessment device according to claim 1, wherein the processing circuitry is further configured to:
assess the cognitive function at a plurality of time points that are different from one another, and
assess a decline in the cognitive function using a change in the cognitive function over the plurality of time points.

4. The cognitive function assessment device according to claim 1, wherein the processing circuitry is further configured to estimate a Mini-Mental State Examination (MMSE) score using an assessment result of the cognitive function.

5. A cognitive function assessment system comprising:
the cognitive function assessment device according to claim 1; and
a stimulus presentation device that presents the stimulus to the subject.

6. The cognitive function assessment system according to claim 5,
wherein the stimulus presentation device is configured to present a visual stimulus, and
wherein the processing circuitry is configured to detect, as the event-related potential, an event-related potential caused by the visual stimulus.

7. The cognitive function assessment system according to claim 5,
wherein the stimulus presentation device is configured to present an auditory stimulus, and
wherein the processing circuit is configured to detect, as the event-related potential, an event-related potential caused by the auditory stimulus.

8. The cognitive function assessment system according to claim 5,
wherein the stimulus presentation device is configured to selectively present a visual stimulus and an auditory stimulus, and
wherein the processing circuitry is configured to:
detect, as the event-related potential, an event-related potential caused by the visual stimulus or an event-related potential caused by the auditory stimulus, and
assess the cognitive function using the event-related potential caused by the visual stimulus or the event-related potential caused by the auditory stimulus.

9. The cognitive function assessment system according to claim 8,
wherein the stimulus presentation device is configured to present a sound for detecting hearing impairment before presenting the auditory stimulus,
wherein the processing circuitry is further configured to:
detect hearing impairment at least using the brain signal, and
when the hearing impairment is detected, send a command that causes the stimulus presentation device to present the visual stimulus,
wherein the stimulus presentation device is configured to present the visual stimulus, and
wherein the processing circuitry is further configured assess the cognitive function using the event-related potential caused by the visual stimulus.

10. The cognitive function assessment system according to claim 5, wherein processing circuitry is further configured to:
estimate, before a stimulus is presented, a possibility of cognitive function decline using a specific frequency of the brain signal, and
assess the cognitive function further using a result of the estimate.
